(19) United States
(12) Patent Application Publication
(10) Pub. No.: US 2008/0082008 A1
(43) Pub. Date: Apr. 3, 2008

(54) Rotatable manometer

(76) Inventor: Hun-Yuan Ke, Taichung (TW)

Correspondence Address: Hun-Yuan Ke;235 Chung-Ho, Box 8-24, Taipei, omitted

(21) Appl. No.: 11/522,805

(22) Filed: Sep. 18, 2006

(51) Int. Cl. A61B 5/02

(52) U.S. Cl. 600/499

(57) ABSTRACT

A rotatable manometer has a manometer body, an electronic device and a tying stripe. A pivotal unit is installed between the electronic device and the tying stripe so that the electronic device is rotatable on the tying stripe. The pivotal unit has a pivotal hole and a pivotal seat. The pivotal hole is formed with a recess and the pivotal seat is formed with a stop block corresponding to the recess; by the engagement of the recess and the stop block, the orientation of the electronic device is adjustable with respect to the tying stripe. Or the pivotal hole has a teethed groove and the pivotal seat is formed with a stop block corresponding to the teethed groove; by the engagement of the teethed groove and the stop block, the orientation of the electronic device is adjustable with respect to the tying stripe.

### FIELD OF THE INVENTION

The present invention relates to manometers, and particularly to a rotatable manometer, wherein the user can rotates the electronic device counterclockwise or clockwise so that the electronic device is rotated to an optimum position. Thereby the operation unit can be operated or viewed,

### BACKGROUND OF THE INVENTION

People often measure blood pressures or pulses by a manometer for recording the values of the blood pressures and pulses for monitoring the conditions of bodies and providing these values to doctors as a reference in treatment. Thus the doctors can adjust the amount of the patient's drugs.

Thus, many people prepare manometers for monitoring blood pressure in a very long term and determine whether it is necessary to go to a hospital for treatment.

The current used manometer is a fixed one, that is, the operation unit and display of the manometer are fixed. The electronic device cannot rotated with respect to the operation unit. Thus in many orientations, the user cannot view the data in the display easily and conveniently. If the user moves the body for viewing the data, the value measured will change with the movement of the body and thus the prior art design is not a preferred one and it is necessary to be improved.

### SUMMARY OF THE INVENTION

Accordingly, the primary object of the present invention is to provide a rotatable manometer, wherein the user can rotates the electronic device counterclockwise or clockwise so that the electronic device is rotated to an optimum position. Thereby the operation unit can be operated or viewed.

To achieve above objects, the present invention provides a rotatable manometer having a manometer body, an electronic device and a tying stripe; wherein a pivotal unit is installed between the electronic device and the tying stripe so that the electronic device is rotatable on the tying stripe. The pivotal unit has a pivotal hole and a pivotal seat. The pivotal hole is formed with a recess and the pivotal seat is formed with a stop block corresponding to the recess; by the engagement of the recess and the stop block, the orientation of the electronic device is adjustable with respect to the tying stripe. Or the pivotal hole has a teethed groove and the pivotal seat is formed with a stop block corresponding to the teethed groove; by the engagement of the teethed groove and the stop block, the orientation of the electronic device is adjustable with respect to the tying stripe.

The various objects and advantages of the present invention will be more readily understood from the following detailed description when read in conjunction with the appended drawing.

### BRIEF DESCRIPTION OF THE DRAWINGS

FIG. 1 is an exploded schematic view of the rotatable manometer of the present invention.

FIG. 2 is an enlarged schematic view about the rotatable manometer of the present invention.

FIG. 3 is a lateral cross sectional view about he rotatable manometer of the present invention.

FIGS. 4 and 5 are schematic views of the rotatable manometer of the present invention.

FIGS. 6 and 7 show the use of the rotatable manometer of the present invention.

FIGS. 8 and 9 are exploded perspective views of the second embodiment of the rotatable manometer of the present invention.

### DETAILED DESCRIPTION OF THE INVENTION

In order that those skilled in the art can further understand the present invention, a description will be provided in the following in details. However, these descriptions and the appended drawings are only used to cause those skilled in the art to understand the objects, features, and characteristics of the present invention, but not to be used to confine the scope and spirit of the present invention defined in the appended claims.

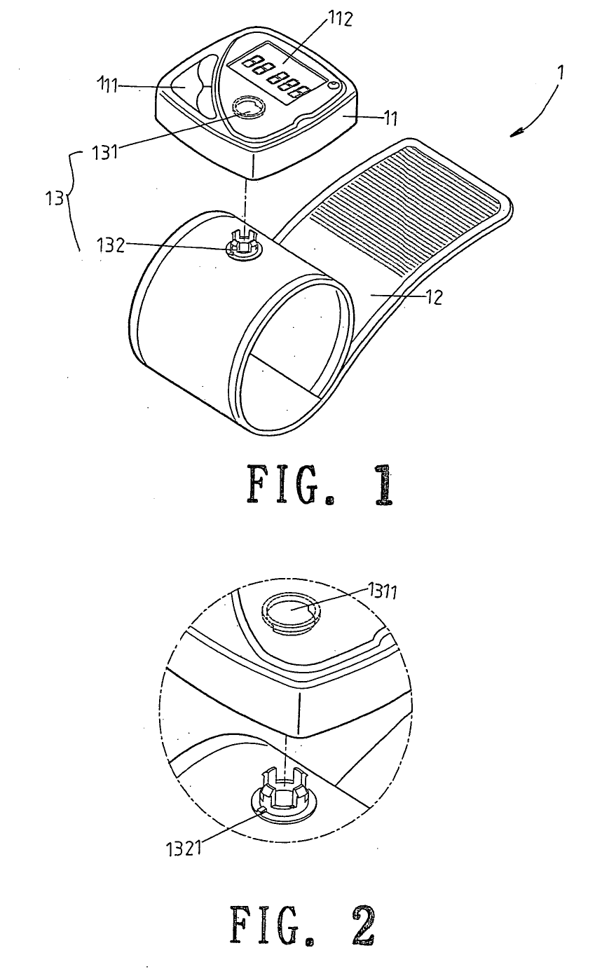

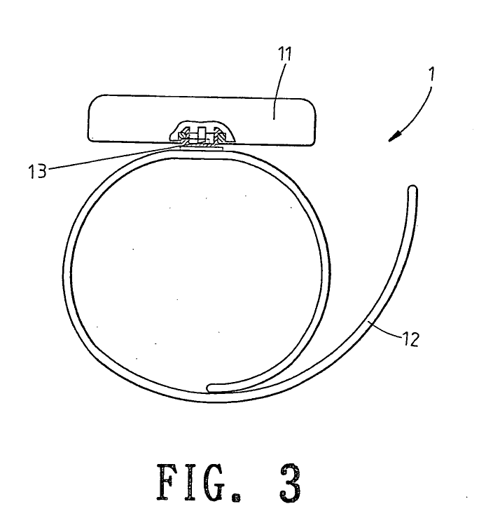

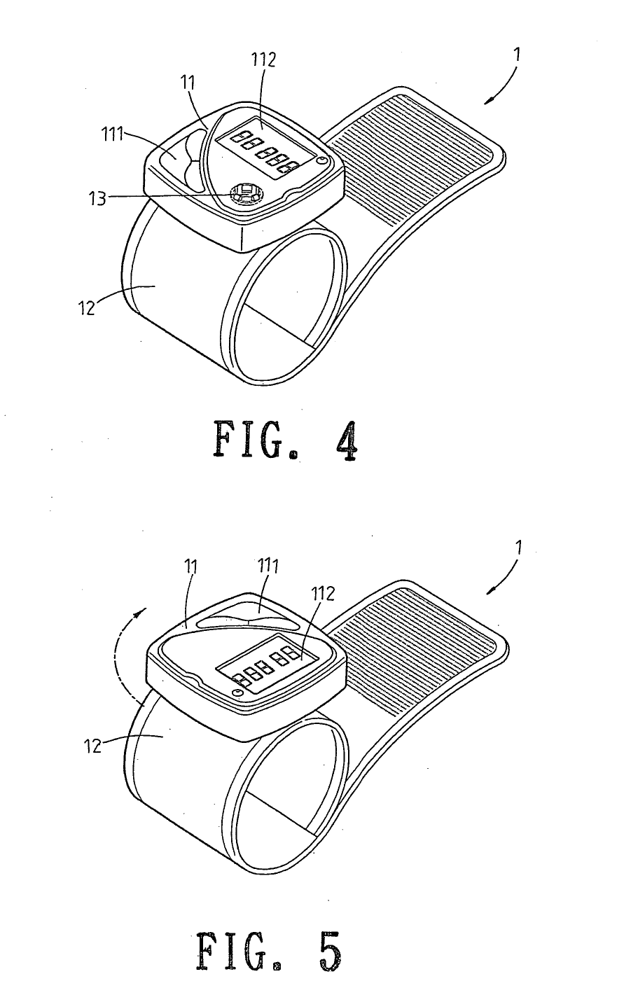

Referring to FIGS. 1 to 5, the rotary manometer of the present invention is illustrated. The present invention has the following elements.

An electronic unit 11 serves to measure the blood pressure and pulses of the blood. The electronic unit 11 has an operation unit 111 and a display 112. The operation unit 111 serves to determine the measuring items about blood pressure and pulses. The measured result is displayed upon the display 112.

The tying stripe 12 can be installed to a measure portion of the user for measuring the blood pressure and pulses (the way for measuring the blood pressure is known in the prior art and thus the details will not be further described herein).

A pivotal unit 13 is installed between the electronic device 11 and the tying stripe 12 so that the electronic device 11 is rotatable with respect to the tying stripe 12. The pivotal unit 13 includes a pivotal hole 131 and a pivotal seat 132 inserted into the pivotal hole 131 tightly. The pivotal seat 132 can be made of plastics. The friction force between the pivotal hole 131 and the pivotal seat 132 serves to adjust the orientation of the electronic device 11 of the manometer body 1.

The pivotal hole 131 is formed at a predetermined position of the electronic device 11. An inner side of the pivotal hole 131 is formed with a recess 1311 of 180 degrees for confining the rotation angle of the electronic device 11.

The pivotal seat 132 is installed on the tying stripe 12. A stop block 1321 corresponding to the recess 1311 is formed on the pivotal seat 132. When the pivotal seat 132 is inserted into the pivotal hole 131, the stop block 1321 is received in the recess 1311. The stop block 1321 only moves within a finite range due to the confinement of the recess 1311.

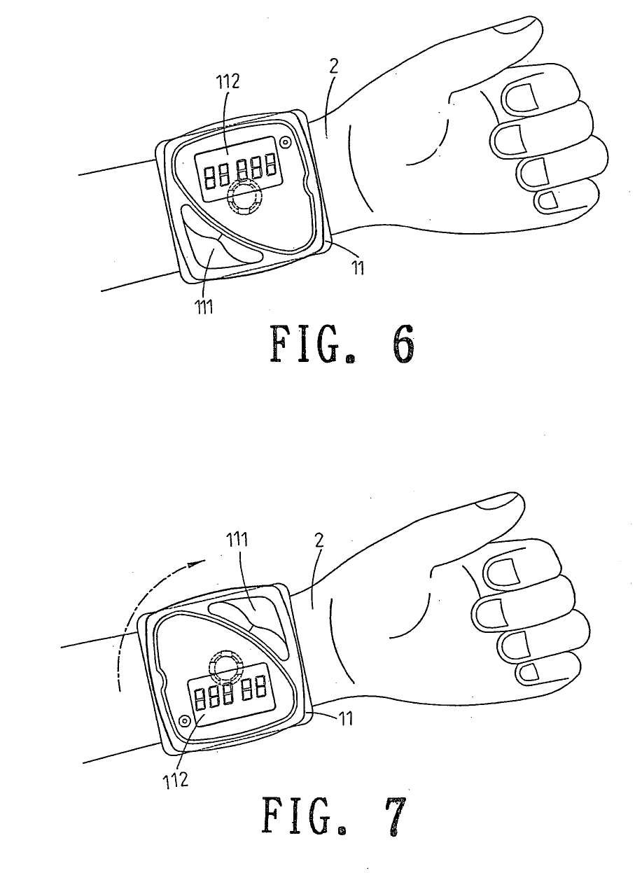

Referring to FIGS. 6 and 7, in use of the present invention, the tying stripe 12 encloses the wrist 2 of the user. The user can rotates the electronic device 11 counterclockwise or clockwise so that the electronic device 11 is rotated to an optimum position. Thereby the operation unit 111 can be operated or viewed.

The rotatable manometer of the present invention can be used to a small size motor so that the electronic device 11 moves automatically. By pressing the operation unit 111, the pivotal unit 13 will rotate to a desired orientation and thus the user can view the display screen 112.

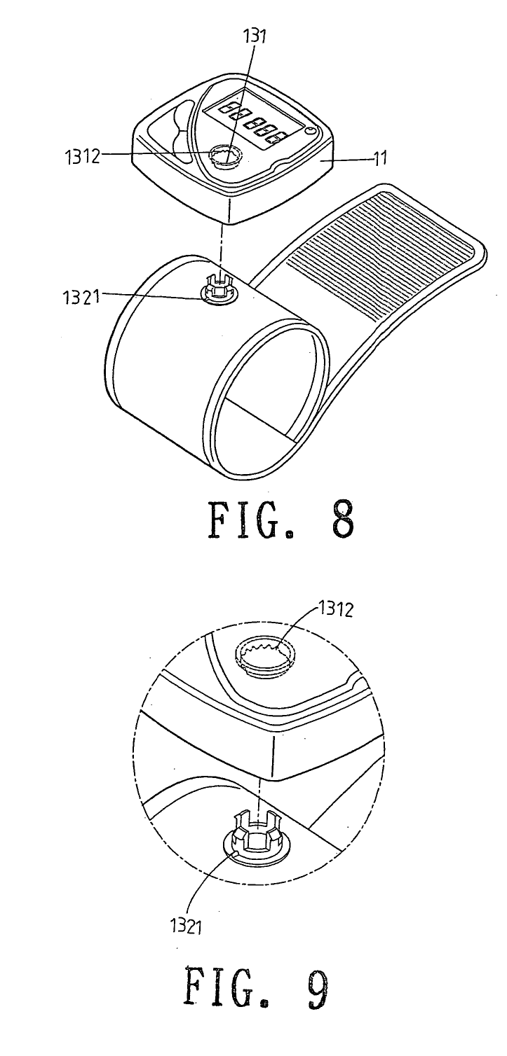

Referring to FIGS. 8 and 9, the second embodiment of the present invention is illustrated. In this embodiment, those identical to the above mentioned embodiment will not be further described herein. Only those difference are disclosed.

In this embodiment, a teethed groove 1312 is installed in the pivotal hole 131. The teethed groove 1312 serves to replace the recess 1311. The pivotal seat 132 is formed with a stop block 1321 corresponding to the teethed groove 1312. The stop block 1321 is a thin strip or a triangular sheet. When the electronic device 11 is rotated, the stop block 1321 will enter into a corresponding teethed groove 1312 so as to be fixed therein. Thereby the orientation of the electronic device 11 is adjustable. If the orientation of the electronic device 11 is shifted again, it is only necessary to rotate the electronic device 11 again, the stop block 1321 will move to other groove.

The present invention is thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A rotatable manometer having a manometer body, an electronic device and a tying stripe; wherein a pivotal unit is installed between the electronic device and the tying stripe so that the electronic device is rotatable on the tying stripe.

2. The rotatable manometer as claimed in claim 1, wherein the pivotal unit has a pivotal hole and a pivotal seat.

3. The rotatable manometer as claimed in claim 2, wherein the pivotal hole is in the electronic device and the pivotal seat is on the tying stripe.

4. The rotatable manometer as claimed in claim 2, wherein the pivotal hole is in the tying stripe and the pivotal seat is on the electronic device.

5. The rotatable manometer as claimed in claim 2, wherein the pivotal hole is formed with a recess and the pivotal seat is formed with a stop block corresponding to the recess; by the engagement of the recess and the stop block, and the orientation of the electronic device is adjustable with respect to the tying stripe.

6. The rotatable manometer as claimed in claim 5, wherein the recess is a cambered recess of 180 degrees.

7. The rotatable manometer as claimed in claim 2, wherein the pivotal hole has a teethed groove and the pivotal seat is formed with a stop block corresponding to the teethed groove; by the engagement of the teethed groove and the stop block, the orientation of the electronic device is adjustable with respect to the tying stripe.

8. The rotatable manometer as claimed in claim 1, wherein the rotation of the pivotal unit is electrically controlled.

9. The rotatable manometer as claimed in claim 1, wherein the pivotal unit is made of rubber which can provides friction force so that the electronic device can be positioned and the orientation of the electronic device is adjustable.
